(19) United States
(12) Patent Application Publication
(10) Pub. No.: US 2006/0029680 A1
(43) Pub. Date: Feb. 9, 2006

(54) Methods of preparing highly concentrated aqueous bromine solutions

(76) Inventors: Michael S. Harvey, Modesto, CA (US); Jonathan N. Howarth, Modesto, CA (US)

Correspondence Address: Audrey A. Millemann;Weintraub Genshlea Chediak Sproul, 400 Capitol Mall, 11th Floor, Sacramento, CA, 95814, US

(21) Appl. No.: 10/912,949

(22) Filed: Aug. 6, 2004

(51) Int. Cl. A61K 33/00

(52) U.S. Cl. 424/600; 424/723

(57) ABSTRACT

The present invention is directed to convenient methods of preparing: (1) highly concentrated liquid bromine-containing biocidal solutions and (2) highly concentrated mixed halogen liquid bromine and chlorine-containing biocidal solutions that have excellent physical and chemical stability. One method involves adding the acidic reaction medium to an alkaline source to effect the final pH adjustment and in another these are co-fed into a common reaction vessel. Both methods minimize the incidence of the acid hydrolysis reaction that undermines chemical yields and generates troublesome sulfate by-products. The methods offer superior reactor cooling efficiencies that reduce batch cycle times and suppress undesirable elevated temperature decomposition reactions.

### BACKGROUND OF THE INVENTION

1. Field of the Invention

The invention relates to methods of preparing highly concentrated liquid bromine-containing biocide solutions and highly concentrated liquid mixed halogen bromine- and chlorine-containing biocide solutions having excellent physical and chemical stability.

2. Description of the Related Art

Single feed bromine-containing biocide solutions are available from a number of sources and many methods to manufacture these products have been reported in the patent literature. These methods fit into two general categories depending on the pH conditions in the early steps of the reaction: those that employ acidic conditions and those that employ alkaline conditions. The present invention addresses the former category.

The methods that employ acidic conditions all share three basic features. First, a source of bromide ion is combined with a nitrogen-containing halogen stabilizer to form a mixture with a pH<7. Second, an oxidizing agent is added to the mixture to oxidize the bromide ion to bromine. Third, an alkaline source is added to the acidic solution to adjust the pH to about 13. This is because the acidic bromine-containing solutions do not possess adequate long term physical or chemical stability.

The acidic condition methods use one of several different oxidizing agents. U.S. Pat. No. 6,270,722 advocates gaseous chlorine or sodium hypochlorite solutions as the oxidizing agent. The use of gaseous chlorine is also taught by U.S. Pat. No. 6,551,624 and U.S. Pat. No. 6,375,991. Ozone is the oxidant described in U.S. Pat. No. 6,007,726. Sodium bromate is the oxidizing agent of choice in U.S. Pat. No. 6,156,229 and U.S. Pat. No. 6,660,307.

U.S. Pat. No. 6,506,418 discusses a problem with the acidic condition methods. The '418 patent, which discloses an acidic method using gaseous chlorine, states that under acidic conditions, “a substantial portion of the sulfamate can be hydrolyzed rather rapidly to sulfate” (col. 3, line 67) and, further, that “loss of sulfamate due to hydrolysis to sulfate can result in decreased storage stability of the finished product [and] imposes an economic burden on the operation” (col. 4, lines 3 and 8). This acid hydrolysis occurs according to the following equation.
2NH₂—SO₃+2H₂O═[NH₄]₂SO₄+H₂SO₄

Thus, in order to overcome the problem, the '418 patent teaches that is desireable to produce solutions with low levels of sulfate, concluding: “if any sulfate is present in the active bromine containing solution as formed, such sulfate content is such that the molar ratio of sulfate to sulfamate is less than about 0.2, and preferably less than 0.05” (col. 4, lines 54-57).

To reiterate, the common feature of all acidic condition methods is the final pH adjustment step that is essential for adequate physical and chemical stability of the products. This step involves the introduction of an alkaline source to the acidic mixture in order to raise the pH to about 13. In all of these prior art methods, sodium hydroxide solution is the preferred alkaline source. It is well known, however, that the addition of an alkaline source causes a significant problem: the acid-base neutralization reaction is strongly exothermic, triggering decomposition reactions that occur at elevated temperatures. These reactions result in lost yield, which is economically disadvantageous, and also result in the formation of an undesirable by-product, sulfate ion, according to the following equation.
2[Br—NH—SO₃⁻]+H₂O═N₂+2H₂SO₄+2Br⁻

The sulfate ion will precipitate as sulfate salts over time, for example, during storage, and can plug pipe work and make feeding of the liquid bromine product mechanically burdensome or impractical. In order to overcome the problems due to the elevated temperature decomposition reactions, the prior art methods require chilling of the reactor contents.

Thus, there is a need to minimize the time that the reaction mixture remains under acidic conditions, as indicated by the '418 patent. The prior art teaches that this need is best addressed by accomplishing the final pH adjustment step quickly. There is also a need, however, to minimize the elevated temperature decomposition reactions that occur when the alkaline source is added, as indicated by the prior art. The prior art teaches that this need is best addressed by efficiently removing heat.

### SUMMARY OF THE INVENTION

The present invention is directed to methods of preparing highly concentrated liquid bromine-containing biocide solutions and highly concentrated liquid mixed halogen bromine- and chlorine-containing biocide solutions that have excellent physical and chemical stability.

The present inventors have discovered that the two needs discussed above (minimizing the time that the reaction mixture remains under acidic conditions and minimizing the elevated temperature decomposition reactions) and the ways in which these needs are addressed by the prior art are in conflict with each other. They conflict because, to address the first problem, the reaction must proceed to a highly alkaline pH as quickly as possible, but, to address the second problem, the addition of the alkaline source used to achieve the high pH must be conducted slowly enough to allow for efficient heat removal.

Thus, there is a need for an effective method of achieving a highly alkaline pH that allows the efficient removal of heat, minimizing both the acid hydrolysis reaction and the elevated temperature decomposition reactions. There is also a need for products with improved physical and chemical stability. The methods of the present invention address these needs.

The prior art teaches that, in methods for making stabilized liquid bromine biocide solutions, the final pH adjustment step is accomplished by using the conventional approach to raising the pH of an acidic medium. That approach is to add an alkaline compound to the acidic medium. The pH of the acidic medium would steadily increase through neutral and into the alkaline region. Addition of the alkaline compound is continued until the desired pH is reached.

Although the approach taught by the prior art appears logical, it has been discovered by the present inventors that the approach is disadvantageous in several respects. The primary reason is that the product is extremely sensitive to the elevated temperature decomposition reactions that occur due to the exothermic nature of the acid-base neutralization reaction. To minimize the elevated temperature decomposition reactions, the bulk of the acidic reaction medium must be chilled. The removal of heat from such a large volume of solution, however, is a function of time for a fixed area of heat transfer surface to volume ratio. Thus, the higher the volume of solution that needs to be cooled, the longer it will take to achieve the targeted temperature. Consequently, manufacturing batch times are prolonged. This is disadvantageous to productivity. Further, the longer the batch remains under acidic conditions, the greater the rate and incidence of the acid hydrolysis reaction listed above. Both the elevated temperature decomposition reactions and the acid hydrolysis reaction reduce yield and produce by-product ions, including sulfate, whose salts tend to precipitate from solution upon storage, detracting from the physical stability of the products.

The methods of the invention include a superior approach to accomplishing the final pH adjustment step, which addresses the problems caused by the acid hydrolysis reaction and the elevated temperature decomposition reactions. The methods may be used to prepare both all-bromine-containing solutions and mixed halogen bromine- and chlorine-containing solutions.

One embodiment of the invention is a method in which the first two steps are conducted under acidic conditions, but the final pH adjustment step, the attainment of a pH of about 13, is conducted in a manner contrary to the conventional approach. In the method of the invention, the acidic medium is added to a solution of an alkaline compound. Thus, the attainment of a highly alkaline pH is approached from the opposite end of the pH spectrum, the alkaline region, rather than from the acidic region. In performing the final pH adjustment step in this manner, an unexpected benefit arises: the efficiency of heat removal, which is crucial to suppress the elevated temperature decomposition reactions, is greatly improved. In cooling the much smaller volume of the solution of alkali, the area of heat transfer surface to volume ratio is much higher. Thus, the lower the volume of solution that needs to be cooled, the less time it will take to achieve the targeted temperature. Consequently, manufacturing batch times are reduced. This is not only advantageous to productivity, but because the batch never passes through the intermediate pH ranges, the incidence of the acid hydrolysis reaction is lower, minimizing lost yield and the formation of sulfate and other by-products.

By adding the acidic medium to a solution of alkali, the pH of the solution does not increase through neutral and into the alkaline region. Instead, the target pH of about 13 is approached from the opposite end of the pH spectrum, to effect not an acid-base neutralization, but a base-acid neutralization. For example, if the pH of an acidic solution is to be raised from a low pH value to 13.5 using a 50% NaOH solution, the conventional approach would be to add 50% NaOH to the acidic solution and monitor the pH through neutral and into the alkaline region until the target pH is reached. In the method of the present invention, the acidic medium is introduced to a sufficient amount of 50% NaOH which is calculated to have an initial pH of 15.6. Consequently, the acidic medium of low pH never passes through the intermediate pH ranges. Instead, the pH decreases from about 15.6 to about 13.5. Of course, many compounds may be present in the acidic medium that might not be able to tolerate such a harsh environment. Unwelcome base-catalyzed hydrolysis reactions might be expected to predominate and destroy products dissolved in the acidic medium. It was discovered, however, that this did not occur with the method of invention: the N-bromosulfamic acid complex present in the acidic medium resists base-catalyzed hydrolytic decomposition reactions. In fact, it was found that N-bromosulfamic acid is remarkably stable to the strongly basic environment of 50% sodium hydroxide solution. Reactions proceeded in essentially quantitative yield, confirming that there was no loss in active ingredient bromine.

Another embodiment of the invention is a method for the production of stabilized bromine biocide solutions in which the first steps are conducted under acidic conditions, but the final pH adjustment step is conducted in an alternative manner that is also contrary to the conventional approach. In this embodiment, the acidic medium and the solution of alkali are co-fed into a common reaction vessel. Again, the acidic medium of low pH never passes through the intermediate pH ranges, but nor does it decrease from pH 15.6. Instead, the rate at which the two solutions are fed together governs the pH of the combination. It is especially convenient to meter the two solutions together so that the starting pH of the combination is between about 10 and about 13.5.

In co-feeding the acidic medium to the solution of alkali, an unexpected benefit arises. While not wishing to be bound by theory, it is believed that the heat removal process, so vital to suppress the elevated temperature decomposition reactions, has much improved efficiency. In cooling the much smaller volume of the solution of alkali, the area of heat transfer surface to volume ratio is much higher. Thus, the lower the volume of solution that needs to be cooled, the less time it will take to achieve the targeted temperature. Consequently, manufacturing batch times are reduced. This is not only advantageous to productivity, but because the batch never passes through the intermediate pH ranges, the incidence of the acid hydrolysis reaction is lower, minimizing lost yield and the formation of sulfate and other by-products.

A further advantage of co-feeding the acidic medium and the solution of alkali is that by continuously withdrawing the reaction products at the same rate as the reactant streams are fed, a steady-state condition develops. Continuous processing allows the reactor size to be significantly reduced without loss in productivity.

The products of the methods of this invention are sources of oxidizing bromine that are useful for microbiological control in aqueous systems. This is generally achieved by introducing the compositions into water requiring microbiological control in an amount sufficient to be biocidally effective. Application areas include a number of industrial water systems such as recirculating cooling water, once-through cooling water, air washer systems, decorative fountains, oil field injection water, oil well completion fluids, municipal and industrial wastewater, brewery pasteurizing water, hydrostatic sterilizer cooling water, pulp and paper processing water, and agricultural irrigation water. Application areas also include a number of residential water systems where the home consumer can apply the compositions in aqueous systems where microbiological control is necessary. Some of these consist of pool and spa water, kitchen and bathroom rinses, toilet bowl rinses, and mold and fungus sprays for inside and outside the home.

### DETAILED DESCRIPTION OF THE INVENTION

This invention is a method of preparing a concentrated liquid bromine-containing biocide composition using a solution of bromide ions and an oxidizing agent. This golden-colored composition contains 50-80% more available bromine than solutions that are currently available commercially. Moreover, the aqueous composition contains the highest concentration of bromine hitherto reported in the prior art. Typically, the composition of this invention contains greater than 18% as Br₂ (8% as Cl₂).

The method of this invention may also be used to prepare a stabilized liquid mixed halogen composition that contains both bromine and chlorine. The method uses a solution of bromide ions in conjunction with a molar excess of a solid organic chlorinating agent. This light golden-colored composition contains 50-80% more available halogen than the all-bromine solutions that are currently available commercially. Typically, the mixed halogen composition prepared using this method contains a total halogen level of greater than 8% expressed as Cl₂ (18% expressed as Br₂).

The method preferably includes the following steps. Steps (a), (b), and (c) may be performed in any order, or simultaneously, followed by the remaining steps, as indicated.

a. Utilizing a Solution of Bromide Ions.

Sources of alkali metal or earth alkali metal solutions of bromide ions include, but are not limited to, lithium bromide, sodium bromide, potassium bromide, calcium bromide, magnesium bromide, and hydrobromic acid. A preferred source of bromide ion solution is sodium bromide solution, commonly available as a 40-46% aqueous solution, or it may be made into such a solution by dissolving solid sodium bromide salt in water.

b. Mixing a Halogen Complexing Agent to the Bromide Ion Solution.

Preferably the complexing agent is sulfamic acid. The amount of sulfamic acid added depends on the amount of bromide ion originally present. A mole ratio of about 0.75:1 to about 1.5:1 sulfamic acid to bromide ions in step (a) is advantageous to the stability of the final product with about 0.95:1 to about 1.2:1 being the most preferred mole ratio range.

c. Adding an Alkaline Source to the Reaction Medium to Adjust its pH to Between about −1 and about +1.

Any alkaline source may be employed. Examples include, but are not limited to, alkali metal or earth alkali metal carbonates, bicarbonates, oxides, and hydroxides. When solutions are preferred, sodium hydroxide or potassium hydroxide solutions are convenient to use, alone or in combination with each other. A particularly preferred alkaline source is 50% NaOH solution. To prevent storage problems in cold climates, 50% NaOH solution may be diluted with water and used. The alkaline source is introduced to the reaction medium slowly, with stirring and cooling, such that the temperature preferably does not exceed about 70° F.

d. Introducing a Bromide Ion Oxidizing Agent to the Reaction Medium.

Suitable bromide ion oxidizing agents include ozone, bromate salts, hydrogen peroxide solutions, and solid organic chlorinating agents. The oxidizing agent is added in an amount sufficient to oxidize all or substantially all of the bromide ions into bromine.

These oxidizing agents are especially convenient bromide ion oxidizing agents because they can be introduced to the reaction medium without proportionally co-feeding a source of alkali for pH control of the reaction medium.

For example, when gaseous Cl₂ is used as the oxidizing agent, the first step involves hydrolysis to form hypochlorous acid and hydrochloric acid.
Cl₂+H₂O=HOCl+HCl
Hypochlorous acid then oxidizes bromide ion to hypobromous acid.
HOCl+Br⁻═HOBr+Cl⁻
Now the requirement for the proportional feeding of a source of alkali becomes apparent. The hydrochloric acid released as shown above must be neutralized.
HCl+NaOH═NaCl+H₂O
This is an absolutely critical step in the reaction sequence. If the HCl was not neutralized, it would accumulate in the reaction medium and the pH would decrease to very low values. Under these conditions, the hydrolysis of Cl₂ as shown above would not occur. Instead, the Cl₂ bubbled into the reaction medium would remain predominantly in the gaseous form and flash from the aqueous phase rendering it unavailable for bromide ion oxidation.

This is in sharp contrast to the situation that occurs when ozone, bromate salts, hydrogen peroxide solutions, and solid organic chlorinating agents are employed as the bromide ion oxidizing agent. For example, in water, trichloroisocyanuric acid (TCCA) hydrolyzes to yield three moles of hypochlorous acid.
TCCA+3H₂O═3HOCl+CA
Hypochlorous acid then oxidizes bromide ion to hypobromous acid.
HOCl+Br⁻═HOBr+Cl⁻

There is no hydrochloric acid co-product, so there is no requirement to neutralize the increased acidity by co-addition of a source of alkali. Indeed, the addition of a source of alkali would soon force the pH into the alkaline region where the oxidation of bromide ion by hypochlorous acid becomes kinetically hindered.

Solid organic chlorinating agents that may be used as a bromine ion oxidating agent include any organic compound in which one or more carbon atoms is present in oxidation state +1 and is covalently bound to a nitrogen or phosphorus atom within the same molecule. Suitable examples include, but are not limited to, trichloroisocyanuric acid (TCCA), sodium dichlorisocyanurate (NaDCC), sodium dichlorisocyanurate dihydrate (NaDCC.2H₂O), potassium dichloroisocyanurate, dichloroisocyanuric acid, trichloromelamine, N-chloro-p-toluenesulfonamide, N-chloromethanesulfonamide, N-chlorosuccinimide, N,N′-1,3-bromochloro-5,5-dimethylhydantoin, N,N′-1,3-bromochloro-5-ethyl-5-methylhydantoin, and 1,3-dichloro-5,5-dimethylhydantoin. A particularly preferred source of a solid organic chlorinating agent is TCCA.

Preferably TCCA is used in the form of a fine granular free-flowing material for ease of introduction to the stirred, cooled reactor. As the TCCA reacts, the coarse granules disappear. The reaction is considered to be complete when no more coarse granules are evident. Although dry, granular TCCA is favored because of its easy handling characteristics, and for providing a visual signal that the reaction is complete, TCCA powdered wetcake may also be employed. The advantage of using TCCA wetcake is that it may be taken directly from the TCCA-producing reactors and thus the costs associated with drying and granulation of the material are eliminated.

In the case where the bromide ion source is a sodium bromide solution, the complexing agent is sulfamic acid, and TCCA is the bromide ion oxidizing agent, the following reaction occurs:
NaBr+NH₂—SO₃H+⅓TCCA→[Br][NH—SO₃H]+NaCl+Cyanuric Acid (1)

In order to prepare a mixed halogen solution, the oxidizing agent used in step (d) must be a solid organic chlorinating agent, such as TCCA. A molar excess of the solid organic chlorinating agent to bromide ions is employed. Employing a 10% molar excess of the solid organic chlorinating agent over the bromide ions yields a mixed halogen composition of 90 mole % bromine and 10 mole % chlorine. In this case, the solid organic chlorinating agent has two functions. First, it oxidizes all of the bromide ions into bromine which reacts with the sulfamic acid to form N-bromosulfamic acid as indicated in reaction (1). Second, the excess chlorinating agent releases soluble chlorine into the aqueous solution by complexing with sulfamic acid to form N-chlorosulfamic acid according to reaction (2).
NH₂—SO₃H+TCCA→[Cl][NH—SO₃H]+Cyanuric Acid (2)

e. Removing any Insoluble Reaction By-Products with a Conventional Solid-Liquid Separation Technique.

Step (e) is required only if the bromide ion oxidizing agent used in step (d) is a solid organic chlorinating agent. If the bromide ion oxidizing agent used in step (d) is ozone, a bromate salt, or a hydrogen peroxide solution, step (e) is not necessary.

Any suitable solid-liquid separation technique can be employed. Suitable techniques include, but are not limited to, centrifugation, clarification, gravity sedimentation, and vacuum filtration. Filtration is a particularly preferred technique for effecting solid-liquid separation.

When the oxidizing agent is TCCA, cyanuric acid (CA) is a reaction by-product that is insoluble in the reaction medium (see reactions (1) and (2)). Filtration of the cyanuric acid residue is carried out at pH −1 to +1, but preferably around pH 0-1.0 to maximize its recovery from solution and minimize the amount of bromine vapors that fume from the reaction medium. Upon washing the filtercake with water to remove the mother liquors, a highly pure CA wetcake is recovered. This wetcake can be recycled to other processes to make additional quantities of TCCA, NaDCC, or NaDCC.2H₂O that can be used in the method of the current invention.

f. Introducing the Reaction Medium into an Aqueous Solution of an Alkaline Source, or Co-Feeding the Reaction Medium and an Aqueous Solution of an Alkaline Source to a Common Junction, such that, in Either Case, the pH of the Combination is at all Times Greater than 7 and Less than a Calculated 15.6.

The alkaline source may be an alkali metal or earth alkali metal hydroxide. Sodium hydroxide or potassium hydroxide solutions are convenient to use, alone or in combination with each other. A particularly preferred alkaline source is 50% NaOH solution. To prevent storage problems in cold climates, 50% NaOH solution may be diluted with water and used. The acidic reaction medium is introduced to the aqueous solution of alkaline source with mixing and with cooling, such that the temperature preferably does not exceed 70° F., and such that the pH of the combination is at all times greater than 7 and less than a calculated 15.6. Alternatively, the acidic reaction medium and the aqueous solution of alkaline source may be co-fed to a common junction with mixing and with cooling such that the temperature preferably does not exceed 70° F. The rate of co-feeding should be such that the pH of the combination is at all times greater than 7 and less than a calculated 15.6.

To prepare the all-bromine-containing liquid composition, if the bromide ion solution is sodium bromide and the alkaline source used in both steps (c) and (f) is an alkali metal hydroxide, the overall mole ratio of bromide ion to hydroxide ion used in steps (c) and (f) is between about 1:2 and about 1:5, preferably between about 1:3 and about 1:4. If the bromide ion solution is hydrobromic acid and the alkaline source is an alkali metal hydroxide, the overall mole ratio of bromide ion to hydroxide ion used in steps (c) and (f) is between about 1:3 and about 1:6, preferably between about 1:4 and about 1:5.

To prepare the liquid mixed halogen composition, if the bromide ion solution is sodium bromide and the alkaline source used in both steps (c) and (f) is an alkali metal hydroxide, the overall mole ratio of chlorine equivalent to hydroxide ion used in steps (c) and (f) is between about 1:2 and about 1:5, preferably between about 1:3 and about 1:4.

In both cases, the purpose of this step is to deprotonate the halo derivatives of sulfamic acid to form the halo derivatives of sodium sulfamate according to reaction (3).
[X][NH—SO₃H]+NaOH→[X][NH—SO₃⁻][Na⁺]+H₂O (3)
X=Br or Cl

g. Removing any Further Insoluble Residues that Develop with a Conventional Solid-Liquid Separation Technique.

Step (e) is required only if the bromide ion oxidizing agent used in step (d) is a solid organic chlorinating agent. If the bromide ion oxidizing agent used in step (d) is ozone, a bromate salt, or a hydrogen peroxide solution, step (e) is not necessary.

As noted above, any suitable solid-liquid separation technique may be employed. Generally, when TCCA is the oxidizing agent, almost 90% of the CA reaction by-product is recovered as a highly pure wetcake in the first solid-liquid separation operation described in step (e). While not wishing to be bound by theory, it is believed that salts of cyanuric acid are precipitated from the reaction medium when it is added to, or co-fed with, the alkaline source in step (f). When the alkaline source is, for example, 50% sodium hydroxide solution, the mono-, di-, and trisodium salts of cyanuric acid are precipitated. Although insoluble in the combination formed in step (f), the di and trisodium salts display increased solubility in ordinary water and are thus useful water treating agents in their own right. However, in comparison to the amount of solids recovered in step (e), the amount of solid that may subsequently develop is relatively low. Thus, step (g) may require only a polishing solid-liquid separation, with, for example, a cartridge filter. If the amount of solid is very low, step (g) may not need to be performed.

### EXAMPLE 1

This example describes the preparation of a mixed halogen composition that contains both bromine and chlorine. A 5% molar excess of solid chlorinating agent over the sodium bromide solution was designed to yield a composition that was 95 mole % bromine and 5 mole % chlorine.

To a stirred reaction flask containing 40% NaBr solution (91.8 g) was added deionized water (15 g) and solid sulfamic acid (42.2 g). The reaction medium was stirred and cooled as a 50% sodium hydroxide solution (30.9 g) was slowly introduced such that the temperature did not exceed 65° F. Finely ground trichloroisocyanuric acid (90% available Cl₂) (29.3 g) was then added to the reaction flask with stirring at such a rate that the temperature did not exceed 66° F. After about 10 minutes, finely ground TCCA was observed to have reacted, as a fine powdery precipitate was observed. Prior to filtration, 50% NaOH solution (3.0 g) was introduced (as a laboratory personnel convenience) to quell the bromine fumes that had developed in the reactor headspace. The filtrate (117 ml) was placed in a dropping funnel that was positioned over an Erlenmeyer flask containing 50% NaOH (40 g) and deionized water (13 g). The flask was cooled and stirred as the contents of the dropper funnel were added at a rate such that the temperature did not exceed 65° F. Immediately upon completing the addition of the acidic bromine-containing solution from the dropper funnel, any additional solids that precipitated from the combined solutions were removed by vacuum filtration. Iodometric titration of the resultant golden-colored solution yielded a total halogen content of 23.35% as Br₂ (or 10.38% as Cl₂). The theoretical amount of Br₂ and Cl₂ equivalent, produced as a function of the amount of TCCA employed, was used to compute a reaction yield of 97.7%.

### EXAMPLE 2

This example describes the preparation of an all bromine-containing composition.

To a stirred reaction flask containing 40% NaBr solution (91.0 g) was added deionized water (20 g) and solid sulfamic acid (41.2 g). The reaction medium was stirred and cooled as 50% sodium hydroxide solution (30.1 g) was slowly introduced such that the temperature did not exceed 67° F. Finely ground trichloroisocyanuric acid (90% available Cl₂) (27.5 g) was then added to the reaction flask with stirring at such a rate that the temperature did not exceed 66° F. After about 10 minutes, finely ground TCCA was observed to have reacted, as a fine powdery precipitate was observed. Prior to filtration, 50% NaOH solution (2.5 g) was introduced to quell the bromine fumes that had developed in the reactor headspace. Upon filtration of the insolubles, the filtercake was washed with two bed volumes of deionized water. The wash liquors were discarded and the filtercake was placed in an oven at 125° F. to dry overnight. The filtrate (124 ml) was placed in a dropping funnel that was attached to one neck of a round bottom flask. The flask was cooled and stirred as the contents of the dropper funnel were co-fed to the round bottom flask with 50% sodium hydroxide solution (15 g) delivered using a syringe. The rate of co-addition was such that the temperature did not exceed 65° F. The pH of the combination did not drop below 11.1 during the co-feeding process. Then, an additional amount of 50% NaOH (30 g) was introduced to the flask. Any additional solids that precipitated from solution were removed by vacuum filtration, immediately upon completing the final addition of 50% NaOH. Iodometric titration of the resultant golden solution yielded a halogen content of 22.29% as Br₂ (or 9.91% as Cl₂). The theoretical amount of halogen produced as a function of the amount of TCCA charged was used to compute a reaction yield of 98.1%. The weight of dry solids removed on the first filtration indicated that 86.2% of the cyanuric acid had been recovered in this step.

### EXAMPLE 3

The chemical stability of the all-bromine formulation prepared in example two was assessed at ambient and elevated temperatures. The sample was poured into a capped plastic container and placed in an oven at 130° F. The amount of active ingredient remaining in the formulation was monitored as a function of time. The physical stability was established by visual observation of whether any solids precipitated from solution over the same period and were evident on the side or bottom of the container, or floating on the surface. The data in Table I shows the results. Even after 26 days at 130° F., less than 25% of the active ingredient was depleted. There was only slight evidence of solids in the elevated temperature sample, and none for the ambient temperature sample.

TABLE I

| | Ambient Temperature | | | 130° F. | | |
|---|---|---|---|---|---|---|
| | | Wt. % | % | | Wt. % | % |
| | | active | Re- | | active | Re- |
| | Solids | ingredient | main- | Solids | ingredient | main- |
| Day | Formed? | as Cl₂ | ing | Formed? | as Cl₂ | ing |
| 0 | No | 9.91 | 100 | No | 9.91 | 100 |
| 10 | No | 9.81 | 99 | Slight | 8.76 | 88.9 |
| 26 | — | — | — | Slight | 7.48 | 75.5 |
| 48 | No | 8.68 | 87.6 | Some | 6.13 | 61.9 |

We claim:

1. A method of preparing a bromine-containing liquid, comprising:
(a) combining a solution of bromide ions, a halogen complexing agent, and a first alkaline source to form a solution;
(b) adding a bromide ion oxidizing agent to said solution; and
(c) introducing said solution to an aqueous solution of a second alkaline source to form a combination, wherein said combination is a bromine containing liquid.

2. A method of preparing a bromine-containing liquid, comprising:
(a) combining a solution of bromide ions, a halogen complexing agent, and a first alkaline source to form a solution;
(b) adding a bromide ion oxidizing agent to said solution; and said solution; and
(c) co-feeding said solution with an aqueous solution of a second alkaline source to form a combination, wherein said combination is a bromine-containing liquid.

3. The method accordingly to claims 1 or 2, wherein said halogen complexing agent is sulfamic acid.

4. The method of claim 3, wherein in step (a), the amount of sulfamic acid added is such that the mole ratio of said sulfamic acid to said bromide ions is between about 0.75:1 and about 1.5:1.

5. The method according to claims 1 or 2, wherein said first alkaline source is selected from the group consisting of alkali metal carbonate, earth alkali metal carbonate, alkali metal bicarbonate, earth alkali metal bicarbonate, alkali metal oxide, earth alkali metal oxide, alkali metal hydroxide, and earth alkali metal hydroxide.

6. The method of claim 5, wherein said first alkaline source is an alkali metal hydroxide, and further, wherein said first alkaline source is 50% sodium hydroxide solution.

7. The method according to claims 1 or 2, where said bromide ion oxidating agent is selected from the group consisting of ozone, bromate salts, hydrogen peroxide solutions, and solid organic chlorinating agents.

8. The method of claim 7, wherein said bromide ion oxidating agent is a solid organic chlorinating agent, and further comprising, after step (b), conducting a solid-liquid separation to remove insoluble by-products from said solution.

9. The method of claim 8, wherein said solid organic chlorinating agent is selected from the group consisting of trichloroisocyanuric acid, sodium dichloroisocyanurate, sodium dichloroisocyanurate dihydrate, potassium dichloroisocyanurate, dichloroisocyanuric acid, trichloromelamine, N-chloro-p-toluenesulfonamide, N-chloromethanesulfonamide, N-chlorosuccinimide, N,N′-1,3-bromochloro-5,5-dimethylhydantoin, N,N′-1,3-bromochloro-5-ethyl-5-methylhydantoin, and 1,3-dichloro-5,5-dimethylhydantoin.

10. The method according to claims 1 or 2, wherein said solution of bromide ions is sodium bromide; said first and second alkaline sources are alkali metal hydroxides; and said bromine-containing liquid has an overall mole ratio of bromide ion to hydroxide ion of between about 1:2 and about 1:5.

11. The method according to claims 1 or 2, wherein said combination formed in step (c) has, at all times, a pH greater than 7 and less than a calculated 15.6.

12. The method according to claims 1 or 2, further comprising, after step (c), conducting a solid-liquid separation.

13. A bromine-containing liquid made in accordance with the method of claim 1.

14. A bromine-containing liquid made in accordance with the method of claim 2.

15. A method of preparing a bromine- and chlorine-containing liquid, comprising:
(a) combining a solution of bromide ions, a halogen complexing agent, and a first alkaline source to form a solution;
(b) adding a bromide ion oxidating agent to said solution; and
(c) introducing said solution to an aqueous solution of a second alkaline source to form a combination, wherein said combination is a bromine- and chlorine-containing liquid.

16. A method of preparing a bromine- and chlorine-containing liquid, comprising:
(a) combining a solution of bromide ions, a halogen complexing agent, and a first alkaline source to form a solution;
(b) adding a bromide ion oxidizing agent to said solution; and
(c) co-feeding said solution with an aqueous solution of a second alkaline source to form a combination, wherein said combination is a bromine- and chlorine-containing liquid.

17. The method accordingly to claims 15 or 16, wherein said halogen complexing agent is sulfamic acid.

18. The method of claim 17, wherein in step (a), the amount of sulfamic acid added is such that the mole ratio of said sulfamic acid to said bromide ions is between about 0.75:1 and about 1.5:1.

19. The method according to claims 15 or 16, wherein said first alkaline source is selected from the group consisting of alkali metal carbonate, earth alkali metal carbonate, alkali metal bicarbonate, earth alkali metal bicarbonate, alkali metal oxide, earth alkali metal oxide, alkali metal hydroxide, and earth alkali metal hydroxide.

20. The method of claim 19, wherein said first alkaline source is an alkali metal hydroxide, and further, wherein said first alkaline source is 50% sodium hydroxide solution.

21. The method according to claims 15 or 16, wherein said bromide ion oxidizing agent is a solid organic chlorinating agent and wherein a molar excess of said solid organic chlorinating agent to bromide ions is employed, and further comprising, after step (b), conducting a solid-liquid separation to remove insoluble by-products from said solution.

22. The method according to claim 21, wherein said solid organic chlorinating agent is selected from the group consisting of trichloroisocyanuric acid, sodium dichloroisocyanurate, sodium dichloroisocyanurate dihydrate, potassium dichloroisocyanurate, dichloroisocyanuric acid, trichloromelamine, N-chloro-p-toluenesulfonamide, N-chloromethanesulfonamide, N-chlorosuccinimide, N,N′-1,3-bromochloro-5,5-dimethylhydantoin, N,N′-1,3-bromochloro-5-ethyl-5-methylhydantoin, and 1,3-dichloro-5,5-dimethylhydantoin.

23. The method according to claims 15 or 16, wherein said solution of bromide ions is sodium bromide; said first and second alkaline sources are alkali metal hydroxides; and said bromine- and chlorine-containing liquid has an overall mole ratio of chlorine equivalent to hydroxide ion of between about 1:2 and about 1:5.

24. The method according to claims 15 or 16, wherein said combination formed in step (c), at all times, has a pH greater than 7 and less than a calculated 15.6.

25. The method according to claims 15 or 16, further comprising, after step (c), conducting a solid-liquid separation.

26. A bromine- and chlorine-containing liquid made in accordance with the method of claim 15.

27. A bromine- and chlorine-containing liquid made in accordance with the method of claim 16.
